(19) United States
(12) Patent Application Publication
(10) Pub. No.: US 2003/0157557 A1
(43) Pub. Date: Aug. 21, 2003

(54) Biocatalysts and method therefor

(75) Inventors: Kerry Kulowski, St. Paul, MN (US); Paul J. Sheldon, St. Paul, MN (US)

Correspondence Address: Mark A. Litman & Associates, P.A., 3209 West 76th St., Suite 205, Edina, MN, 55435, US

(73) Assignee: Acera Biosciences, Inc.

(21) Appl. No.: 10/082,661

(22) Filed: Feb. 20, 2002

(51) Int. Cl. G01N 33/53; C12P 21/06; C12N 15/85; G01N 33/543

(52) U.S. Cl. 435/7.1; 435/69.1; 435/455; 436/518; 435/189; 435/228

(57) ABSTRACT

One or more biocatalysts or a combinatorial array of biocatyalysts is produced by providing a host cell; recombining at least two biotransformation genes for modifying a chemical substrate into the host cell; and thereby producing at least one recombinant strain comprising a biocatalysts. Additionally, the screening of cells having at least two distinct biotransforming catalysts combined therein may be performed by providing multiple wells, each of the multiple wells containing cells having at least two distinct biotransforming catalysts combined therein; and providing multiple wells, each of the wells containing different cells having at least two distinct biotransforming catalysts combined therein; and then allowing cells within the well to catalyze a reaction with a compound within each well, and analyzing the results of any reactions within each well to determine any functional activity of cells within each well.

### FIELD OF THE INVENTION

This invention relates to biocatalysts produced by genetic recombinations in a host cell, the synthesis of biocatalysts and the process of using host cells to produce biocatalysts. The biocatalysts are useful to modify chemical substrates.

The strategy of analyzing microbial cultures for new products after adding an exogenous (i.e., not produced by the native culture) chemical substrate is known as biotransformation screening. This method, which has been used for several decades, is usually performed using actively growing, pure cultures from a culture collection. Microbial biotransformation methods have been shown to perform nearly every chemical conversion known. Examples of processes using whole cells resulting from screening processes include producing steroidal hormones from abundant precursors, microbial modeling of mammalian drug metabolism, and extensions of these approaches (e.g., Smith, R. V. and Rosazza, J. P., Microbial models of mammalian metabolism. J. Nat. Prod., 1983, 46, 79-91; and Roberts, S. M. et al., Introduction to Biocatalysis Using Enzymes and Micro-organisms, Cambridge: Cambridge University Press; 1995, 147-153.

The screening techniques that are generally used involve taking a single chemical and placing that chemical into a series of reaction vessels with numerous pure cultures (e.g., up to about 100) of different types of microbial cells. The purpose of the screen is to find one or more strains from a culture collection with an ability to transform the chemical into a desired product.

Gene replacement by addition has been used for introduction of a biosynthetic gene (or “evolved” gene) into a host possessing an existing biosynthetic pathway or partial pathway. Madduri et al., in “Production of the antitumor drug epirubicin (4′-epidoxorubicin) and its precursor by a geneticially engineered strain of Streptomyces peucetius, Nat. Biotechnol.” 1998, 16, 69-74, disclosed making epirubicin using a mutant doxorubicin production strain transformed with a deoxysugar gene from another antibiotic biosynthetic pathway. A variation of this approach is to evolve an enzyme for a specific activity and then incorporate this activity into the pathway by addition or replacement of the modified gene responsible for the specific enzyme activity (e.g., May et al., “Inverting enantioselectivity by directed evolution of hydantoinase for improved production of L-methionine, Nat. Biotechnol.” 2000, 18, 317-320).

Combinatorial biosynthesis is well known in the art and relates to recombination of biosynthesis genes to create new biosynthetic pathways in a host leading to hybrid structures (e.g., Hutchinson, C. R., “Combinatorial biosynthesis for new drug discovery.” Curr. Opinion Microbiol. 1998, 1, 319-329; and European patent specification EP 0 725 778).

Mutasynthesis, also called mutational biosynthesis, is a known process of blocking a gene or genes early in the pathway (i.e., starter units) and “feeding” unnatural substrates to this mutant to be processed normally by the remainder of the biosynthetic pathway (e.g., Dutton, C. J. et al., “Novel avermectins produced by mutational biosynthesis. J. Antibiot.” 1991, 44, 357-365).

Combinatorial biocatalysis relates to a known process for using enzymes and microbes for iterative derivatization of small-molecule lead compounds and is disclosed in U.S. Pat. No. 6,136,961 (Dordick, J. S. et al., October 2000). No genetic component is used in this technology.

Random recombinant enzyme libraries generated from pools of cDNA and genomic DNA fragments from microorganisms and environmental samples when expressed have been described for preparation of enzyme kits and enzyme libraries as disclosed in U.S. Pat. No. 6,004,788 (Short, J. M., December 1999). This work represents a method for screening for oxidoreductase, transferase, hydrolase, lyase, isomerase, and ligase activity.

### BRIEF DESCRIPTION OF THE INVENTION

The present invention relates to a genetic approach for using microbes (whole cells) for iterative biotransformations within a host cell containing a gene expression cassette of one or more genes behind a promoter sequence. The microbes contain engineered gene expression cassettes that produce high levels of catalytic or enzymatic moieties that can be used to control desired organic reactions and effect desired chemical changes in reagent materials. Enzymes are generally known in the biochemical art as highly selective catalysts. Their hallmark is the ability to catalyze reactions with exquisite stereo-, regio-, and chemo-selectivities in contrast to conventional synthetic chemistry methods which usually requires multiple step reactions to achieve the same outcome. Moreover, enzymes are remarkably versatile. They can be tailored to function in organic solvents, operate at extreme pH's and temperatures, and catalyze reactions with compounds that are structurally unrelated to their natural, physiological substrates. Enzymes also tend to be reactive toward a wide range of natural and unnatural substrates, thus enabling the modification of virtually any organic reagent compound. Moreover, unlike traditional chemical catalysts, enzymes are highly enantio- and regio-selective. The high degree of functional group specificity exhibited by enzymes enables one to keep track of each reaction in a synthetic sequence leading to a new active compound. Enzymes are also capable of catalyzing many diverse reactions unrelated to their physiological function in nature. For example, peroxidases catalyze the oxidation of phenols by hydrogen peroxide. Peroxidases can also catalyze hydroxylation reactions that are not related to the native function of the enzyme. Other examples are proteases which catalyze the breakdown of polypeptides. In organic solution, some proteases can also acylate sugars, a function unrelated to the native function of these enzymes.

### DETAILED DESCRIPTION OF THE INVENTION

Within one aspect of the invention, genetically transformed cells exploit the unique catalytic properties of enzymes resulting from expression of catalytic genes or gene segments. Whereas the use of biocatalysts (i.e., purified or crude enzymes, non-living or living cells) in chemical transformations normally requires the identification of a particular biocatalyst that reacts with a specific starting compound, the present invention may use selected biocatalysts and reaction conditions that are specific for functional groups that are present in many starting compounds. Each biocatalyst may be specific for one functional group, or may be more generally effective for several related or relatively unrelated functional groups. The biocatalyst may remain intact through the intended biochemical reaction or may react with starting compounds containing this functional group. Although this is not within the classic definition of catalysts, it is within the scope of the present invention, whether or not the reacted group of the catalyst may be independently regenerated or returned to its original reactive state so that the cell may be reused.

The biocatalytic reactions can be used to produce a population of derivatives from a single starting compound or a single species of derivatives. These derivatives can be subjected to another round of biocatalytic reactions (e.g., an iterative reaction in the same medium or in a separate medium) to produce a second population of derivative compounds. Thousands of variations of the original compound or a distinct line of variation (that is, a single end product, with or without byproducts) can be produced with each iteration of biocatalytic derivatization.

More specifically, the enzymatic reactions may be conducted with a group of transferred or inserted catalyst or enzyme sequences encoded in the gene expression cassette, creating a cell that reacts with distinct structural moieties found within the structure of a starting compound. Each enzyme or catalyst structure within the microbe or cell may be specific for one structural moiety or a group of related structural moieties. Furthermore, each enzyme reacts with many different starting compounds that may contain the distinct structural moiety.

Briefly, the present invention relates to a method for producing a combinational array of biocatalysts,a plurality of combinational arrays of biocatalysts, the method comprising the step of providing a host cell comprising at least one recombination of a biotransformation gene or multiples of biotransformation genes (or gene sequences, hereafter collectively referred to as ‘genes’). The biotransformation genes are provided into the cell or microbe by a standard microbial transformation method (e.g., chemical or physical methods) so that they may modify a chemical substrate. The effect of the provision of the multiples of biotransformation genes into the cell or microbe is to produce one or more combinatorial arrays of recombinant strains, the strains comprising a functional biocatalyst. At least one of the biotransformation genes expressed in the host should be active in a desired reaction path imposed on a reagent to be catalyzed or enzymatically directed by the biocatalyst.

The biocatalysts (enzymes) are produced by overexpression of single or multiple genes in a whole cell. The biotransformation genes can be from diverse sources, primarily biosynthetic pathways from microorganisms. The recombining of multiple genes from classes of similar enzymes (e.g., four different monooxygenases), or from classes of dissimilar enzyme activities (e.g., two different monooxygenases and two different methyltransferases) to create unique biocatalysts is not known. The process for incorporating any individual genes or gene sequences into a cell or microbe is well within the skill of the artisan, as is the ability to incorporate multiple genes or multiple gene sequences into a cell or microbe. The concept of combining or incorporating such multiple genes or multiple gene sequences into a single cell or microbe has not been disclosed prior to this invention. Therefore, the ability of the skilled artisan to synthesize the biocatalysts of the present invention is within the skill of the artisan once motivated by the concept and instruction of the present invention.

The present invention biocatalyst(s) and its attendant potential and exercise as a biotransformation process uses microbial host cells to efficiently take up and modify chemical substrates or reagents added to the preferred format of resting cell preparations (i.e. cells not growing, but with biotransformation enzymes and cofactors intact) containing gene expression cassettes which give rise to high levels of biocatalysts (i.e., biotransformation enzymes). The biotransformation biocatalysts of the present invention differ at least in part from previous wild-type or recombinant whole cell biocatalysts by having incorporated therein specific recombinations of multiples of biotransformation genes (e.g., at least two different biotransformation genes, preferably at least three different biotransformation genes; by way of further example, a series of at least two different monooxygenase genes) in a single host. The engineered, recombinant strains of the present invention comprising biotransformationally effective biocatalysts have the capacity to generate libraries of single analogs or many new analogs from a single substrate (e.g., a natural product or xenobiotic chemicals). By analyzing a data matrix that compares new analogs to gene combinations, identification of genes and their resulting metabolites is possible.

Conventional whole cell biotransformation methods typically lack high throughput capacity for new analog generation because each metabolite profile must be recovered for addition to subsequent screening rounds, an inefficient and expensive process. In contrast, the present invention process allows for the possibility and enables many possible analogs being formed from a single round of contact with the microbial biocatalyst array described because this process may generate a large number of recombinant strains (each potentially a unique biocatalyst) containing unique combinations of many modifying genes into an expression cassette. The present invention using biotransformation genes can be derived from whole cells endowed with biotransformation ability as a result of genetic recombination and in vivo expression from one or both of constitutive promoter(s) and inducible promoter(s) to create whole-cell biocatalysts. In this format the possible number of unique biocatalysts represented by the potential biotransformation activities encoded by two or more genes is indicated in the formula (2ⁿ−n−1), where n=the number of unique gene sequences. As an example, a series of gene expression cassettes created from 5 genes expressed in a host cell would give rise to 26 unique enzyme combinations (i.e., 10 combinations of 2 genes, 10 combinations of 3 genes, 5 combinations of 4 genes, and 1 combinations of 5 genes). It is clearly possible by the practice of the present invention to provide an appropriate combination of genes into a single microbe or cell to perform a biotransformation capability along a specific sequence of steps, not merely to perform a single step. For example, three distinct genes may be provided that at least some of the genes catalyze the performance of a precursor step with respect to the step to be catalyzed by another gene or gene combination expressed within the same cell. It is also possible for mixtures of cells with transformed genes to be provided in a single environment so that sequential steps may be performed by one or more cells at a time. By way of example only, a first gene or gene combination may catalyze the formation of an alcohol on a specific type of group. A second gene or combination of genes may catalyze the formation of a carboxylic acid, and a third gene or set of genes or gene combinations may catalyze a the formation of an ester. In this manner, as one form of practice of the invention, each modification (new product) could then be a substrate for another enzyme; the actual sequence of activities could be different for different substrates. It also follows that an O-methyltransferase would need an oxygen which would have to be introduced by an oxygenase, if the substrate did not have free hydroxyl (or N, or S functionality for other kinds of transferases). The traditional methods of iterative biotransformations involve isolation of a new product followed by another round of biotransformation with that new product. An approach published by Albany Molecular Inc. (formerly Enzymed), uses commercially-available, purified enzymes, or whole cells, and then moves the liquids around by hand or with robotics for subsequent biotransformation steps. In one aspect of the present invention it is possible to do all the possible transformations in the array of cells, and using commercially unavailable enzymes engineered into the host through expression of genes from sequenced biosynthetic pathways. By using bioinformatics (comparing sequence to function) one may a priori speculate about the biocatalytic activity of a gene by looking at the structure of the endogenous metabolite associated with the biosynthetic pathway. In this way, it is possible to intelligently choose genes which modify structures, like cytochrome P450s, peroxidases, dioxygenases, methyltransferases, glycosyltransferases, reductases, and the like. When the arrays of expressed genes are challenged with exogenous substrates, an array of modified structures may be expected. Using this new system will assessment of single or multiplebiotransformation possibilities with one round of incubations (e.g., at least 2 wells, at least 12 wells, at least 20 wells, at least 24 wells, at least 40 wells, at least 48 wells, possibly in 96 wells or more, e.g., at least 100 wells, at least 200 wells, or a continuous series of wells). Also, because the genetic makeup of the gene expression cassettes is known, a correlation between products obtained and gene combination one should be able to determine which gene sequence is responsible for a given transformation. Presently there is no disclosure in the art of mixing and matching an array of genes in this way to carry out biotransformations. One aspect of the present invention resides in the use of single host strains containing two or more enzymes or overexpressed enzymes associated with cell metabolism or from secondary metabolism pathways (e.g., antibiotic pathways) including oxidoreductases, transferases, hydrolases, lyases, isomerases, and ligases, and similarly overexpressed cofactor regeneration enzymes, when required. The term “overexpressed” is typically used in the art to define the presence of an expression cassette comprised of a gene and promoter in a cell or other carrier that allows the cell or carrier to express an enzyme at an elevated level because of the presence of the promoter sequence and/or resulting from copies of the gene at levels in excess of that normally found in that cell or other cells. Each strain (same host, with a unique genetic make-up) has the potential of generating singly- or multiply-modified substrates. Overexpressed genes in resting cells (i.e., having biochemical potential, yet not growing) will dominate any biochemical activity well above baseline activity, leading to accumulation of metababolites over time. This effect can be analogized to turning up the volume of the biocatalyst due to overexpression of the gene, while minimizing the potentially competing activities of enzymes present in growing cells (e.g., enzymes that might degrade the added substrate and possibly use it as a growth substrate).

In addition, the present invention uses engineered gene cassettes with two, or more consecutive genes (these may be consecutive in physical alignment and/or consecutive within the sequence of reactions or chemical steps that can be catalyzed by the specific genes), behind a strong promoter in an expression vector specific to a host cell. The genes can be isolated from a variety of sources including biosynthetic pathways for natural products, and then recombined using general molecular biology methods. When the resulting expression cassette (e.g., a multiplicity of genes, such as at least two genes, at least three genes, at least four genes, etc. are provided in a single packet (i.e. as the engineered expression vector), and that packet or cassette may be inserted into various cells or microbes) is transformed into the host and maintained by selection, and/or chromosomal integration, and a new strain with unique biotransformation potential is produced. These strains are grown using conditions for optimal promotor activity, harvested by centrifugation or filtration, resuspended in optimal buffer along with a chemical substrate, and analyzed for new product(s).

Another feature of this invention is the discovery that in the absence of endogenous substrate and regulation controls, recombinant genes from a nonessential metabolic pathway, when overexpressed in a heterologous host, may show broader substrate capacity. We have found that enzymes associated with a biosynthetic pathway are regulated by the host (i.e., expressed only at certain times in the life cycle, under strict control). Furthermore, when the endogenous substrate is present in the producing microbe (i.e., the physiological or “correct” substrate), there is little opportunity for an exogenous substrate to be transformed because of competition by the correct substrate. By removing controls (i.e., moving to another host cell, removing gene regulation, by expressing the gene to high levels, and by using resting cells), the added foreign substrate may then become a substrate subject to modification.

One method for producing a combinatorial array of biocatalysts comprises the steps of providing a host cell; recombining one biotransformation gene for modifying a chemical substrate into the host cell; thereby producing at least one recombinant strain comprising a biocatalyst; then inserting the at least one recombinant strain comprising a biocatalyst into at least two sections of an array of biocatalysts. This method may have the at least one biotransformation gene introduce a chemical functional group selected from the group selected from carbon to carbon bonds, hydroxylation, halogenation, cycloaddition, and amination.

In a narrower consideration of the present invention, it is possible for the identification of a gene or set of genes to perform a desired biotransformation process, including but not limited to a) introduction of chemical functional groups to provide formation of carbon-carbon bonds, hydroxylation, halogenation, cycloadditions, and amination, b) modification of existing functional groups to provide reduction of carboxylic acids, aldehydes, and ketones; oxidation of alcohols, sulfides, amino groups, and thiols; hydrolysis of nitrites; replacement of amino groups with hydroxyl groups; ring cyclization; isomerization; epimerization; dealkylation; and c) functional group addition such as acylation, glycosylation, amidation, phosphorylation, and alkyl (e.g., methyl) transfer. It is therefore within the scope of some aspects of the invention to perform a screening process to identify functions within genes and multiple gene sequences (e.g., cassettes). The functions to be identified would be novel functions on compounds or groups, which functions were not necessarily anticipated or expected from those groups or cassettes. For example, we might find that some genes encode enzymes that have a different function than we expected, even when some functions of the enzyme are known. An example would be cytochrome P450s (monooxygenases) which often introduce oxygen as a hydroxyl group at a carbon atom. This class of enzyme may also perform other reactions such as O-dealkylation and dehalogenation reactions—the functionality possibly depending on the substrate. Thus a goal of this biotransformation methodology is to generate new analogs and later identify the gene or set of genes which may be correlated with a new product(s).

Useful chemical substrates (molecules) for the present invention include natural products such as terpenes like monoterpenes and diterpenes (e.g. plant oils, steroids); polyketides (macrolides and polyaromatics); alkaloids; as well as natural or synthetically-derived bioactive chemicals like vitamins; hormones; anticancer agents; antibiotic agents; pesticides; and synthetic or biosynthetic intermediates of these various substances.

Examples of biocatalysts of the invention include but are not limited to monooxygenases; dioxygenases; methyltransferases; and glycosyltransferases.

Examples of recombinant strains of the invention include the following example of using three unique genes to make seven new strains: PikC; MitM; MmcS; PikC with MitM; PikC with MmcS; MitM with MmcS; PikC with MitM with MmcS; where PikC is a cytochrome P450 (from Streptomyces venezuelae, Xue, Y., et al. Hydroxylation of macrolactones YC-17 and narbomycin is mediated by the PikC encoded cytochrome P450 in Streptomyces venezuela. Chem. Biol., 1998, 5, 661-667). MitM is a methyltransferase (from Streptomyces lavendulae), and MmcS is a carbamoyl transferase (also from Streptomyces lavendulae; Mao, Y. et al. Molecular characterization and analysis of the biosynthetic gene cluster for the antitumor antibiotic mitomycin C from Streptomyces lavendulae NRRL 2564, Chem. Biol., 1999, 6, 251-263).

As an example of the process for the invention we propose to use Streptomyces lividans as a general host strain for overexpression of combinations of biocatalytic genes. The genetics and methodologies for introducing expression vectors into protoplasts of streptomycetes as recombinant hosts, and plasmid maintenance using antibiotic selection markers, are well known to those skilled in the art (detailed procedures in Hopwood, D. A., et al. Genetic manipulation of Streptomyces: a laboratory manual; 1985, 103-114.)

Construction of a Series of Expression Vectors using Three Different Genes

Construction of a series of expression vectors relies on standard DNA cloning and DNA sequencing methods well known to those skilled in the art. In this example using three unique genes (a gene encoding a cytochrome P450 (cytP450I), a gene encoding O-methyltransferase (OMT), and a gene encoding a second cytochrome P450 (cytP450II), each with engineered cloning sites by using the polymerase chain reaction (PCR)), a series of seven expression vectors (with one control vector) would thus be constructed (see table of plasmids, #1-8).

The first step for this example is insertion of a Streptomyces promoter into a Streptomyces shuttle vector by using the unique restriction sites BamHI and EcoRI in the polylinker region to make Plasmid 1 (note: the names of restriction enzymes, like BamHI, are often used to indicate a restriction site in a sequence of DNA). Plasmid 1 would then be digested with XbaI and HindIII (present in the polylinker downstream of the promoter) for ligation of cytochrome P450I gene (engineered using PCR with flanking XbaI and HindIII sites) to make plasmid 2. Similarly, plasmid 1 would be digested with polylinker sites BamHI and XbaI for insertion of, OMT gene to make plasmid 3, and similar insertion of cytP450II into plasmid 1 to make plasmid 4. Plasmids 5-7 each containing two different genes would be made as follows. Plasmid 5 and plasmid 6 would each be created by starting with plasmid 2 digested with polylinker sites BamHI and XbaI followed by respective insertion of OMT and cytP450II genes. Plasmid 7 would be created by starting with plasmid 3 digested with polylinker sites BamHI and SpeI followed by insertion of cytP450II gene as a BamHI and XbaI fragment (note: XbaI and SpeI are compatible DNA single strand sequences resulting from restriction, i.e. compatible ends). Finally, plasmid 8 would have all three genes by digesting plasmid 5 with BglII and XbaI and inserting cytP450II gene as a BamHI and XbaI fragment (note: BglII and BamHI are compatible ends). When all of the plasmids are thus completed and verified by restriction analysis, each would then be transformed in parallel into protoplast preparations of a Streptomyces host and selected with antibiotic to isolate transformants (detailed procedures in Hopwood, D. A., et al. Genetic manipulation of Streptomyces: a laboratory manual; 1985, 103-114.) The resulting colonies for the eight genes would give eight unique strains, seven of which would be grown for use as biocatalysts (possible uses indicated in table 1). A flow chart example of a monoterpene substrate (circled structure) and a range of possible products resulting from biotransformation procedures using strains created from each of the completed plasmids is shown in scheme 1. Scheme 1 depicts several products formed from a spontaneous dehydration (i.e. loss of H and OH to give a double bond moiety), many other plausible structures are not depicted, and furthermore not all of the depicted structures would likely be formed from this exemplary set genes. A larger biocatalysis array (i.e. more genes in more combinations) would be capable of generating a larger library of products.

TABLE 1
The example of three genes engineered and recombined
to create an array of seven biocatalysts equal to
2ⁿ- 1; four multiple gene biocatalysts
equal to 2ⁿ-n-1, where n = number
of genes.

| Vector (V) + | | | |
|---|---|---|---|
| additional | Plasmid | Flanking sites | Intended use |
| DNA | designation | (additional sites) | as biocatalyst |
| V + | Plasmid 1 | EcoRI | control |
| promoter (p) | (no gene) | BamHI | |
| Plasmid 1 + | Plasmid 2 | XbaI | Insertion of oxygen at |
| cytP45OI | (1 gene) | HindIII | existing carbon |
| Plasmid 1 + | Plasmid 3 | BamHI(SpeI) | Methyl addition to existing |
| OMT | (1 gene) | (Bgl/II)XbaI | oxygen |
| Plasmid 1 + | Plasmid 4 | BamHI(SpeI) | Insertion of oxygen at |
| cytP450II | (1 gene) | (Bgl/II)XbaI | existing carbon |
| Plasmid 2 | Plasmid 5 | BamHI(SpeI) | Methyl addition to |
| +OMT | (2 genes) | (Bgl/II)XbaI | new oxygen |
| Plasmid 2 + | Plasmid 6 | BamHI(SpeI) | Multiple insertion of |
| cytP450II | (2 genes) | (Bgl/II)XbaI | oxygen at existing carbons |
| Plasmid 3 + | Plasmid 7 | Bgl/II | Methyl addition to new |
| cytP450II | (2 genes) | XbaI | oxygen |
| Plasmid 5 + | Plasmid 8 | BglII | Multiple methyl addition to |
| cytP450II | (3 genes) | XbaI | new oxygen atoms |

Biotransformation Procedures

Many of the specific protocols will depend on the nature of the biotransformation, especially the physical and chemical properties of the chemical substrate and expected biotransformation products. A general description of methods for use of this invention follows.

Each biocatalyst (control, or expression strain) is grown using standard fermentation conditions for streptomycetes (shake flask, or fermenter, in a suitable growth medium containing appropriate selection pressure (e.g. antibiotic)). Upon reaching a suitable cell density (strain dependent, e.g. 24-120 hours), cell mass would be recovered by centrifugation and spent growth medium would be removed by decanting or aspiration. Wet cell mass is resuspended in a suitable buffer (Tris, pH 7.5 or Phosphate, pH 7.4) and subjected to a rinse step by centrifugation and buffer removal. Cells would then be respuspended in buffer and stored cold or frozen (including cryoprotectant such as 15%, glycerol) until material is needed for a biotransformation study with chemical substrate(s). The latter preparation of stored cells is known as a resting cell preparation. Resting cell preparations are biocatalytically active, however they are not actively growing and dividing.

Aliquots of each biocatalyst are resuspended in individual flasks, or wells of multi-well plates using an appropriate biotransformation buffer. A given substrate, such as the monoterpene shown in scheme 1, is added neat, or as a liquid or solid preparation, (e.g. DMF solution, slurry, etc.). The quantity of substrate added will vary depending on the amount of biocatalyst used and/or detection limits of substrate and/or product(s). Flasks or plates would then be allowed to incubate under appropriate biotransformation conditions (temperature, humidity, atmosphere (oxygen, etc.)). At single or multiple time points, flasks or wells are sampled and subjected to analysis (chemical or biological) to determine the presence of new metabolites/products. Profiles of products corresponding to strains tested should reveal which gene or combination of genes encodes the biocatalyst(s) responsible for the observed biotransformation. A time course study may be needed to observe the full range of possible products in some cases. As in scheme I, a late time point analysis may efficiently produce a series of multiply-modified structures, whereas an early time point analysis may reveal an abundance of an earlier singly-modified structure series.

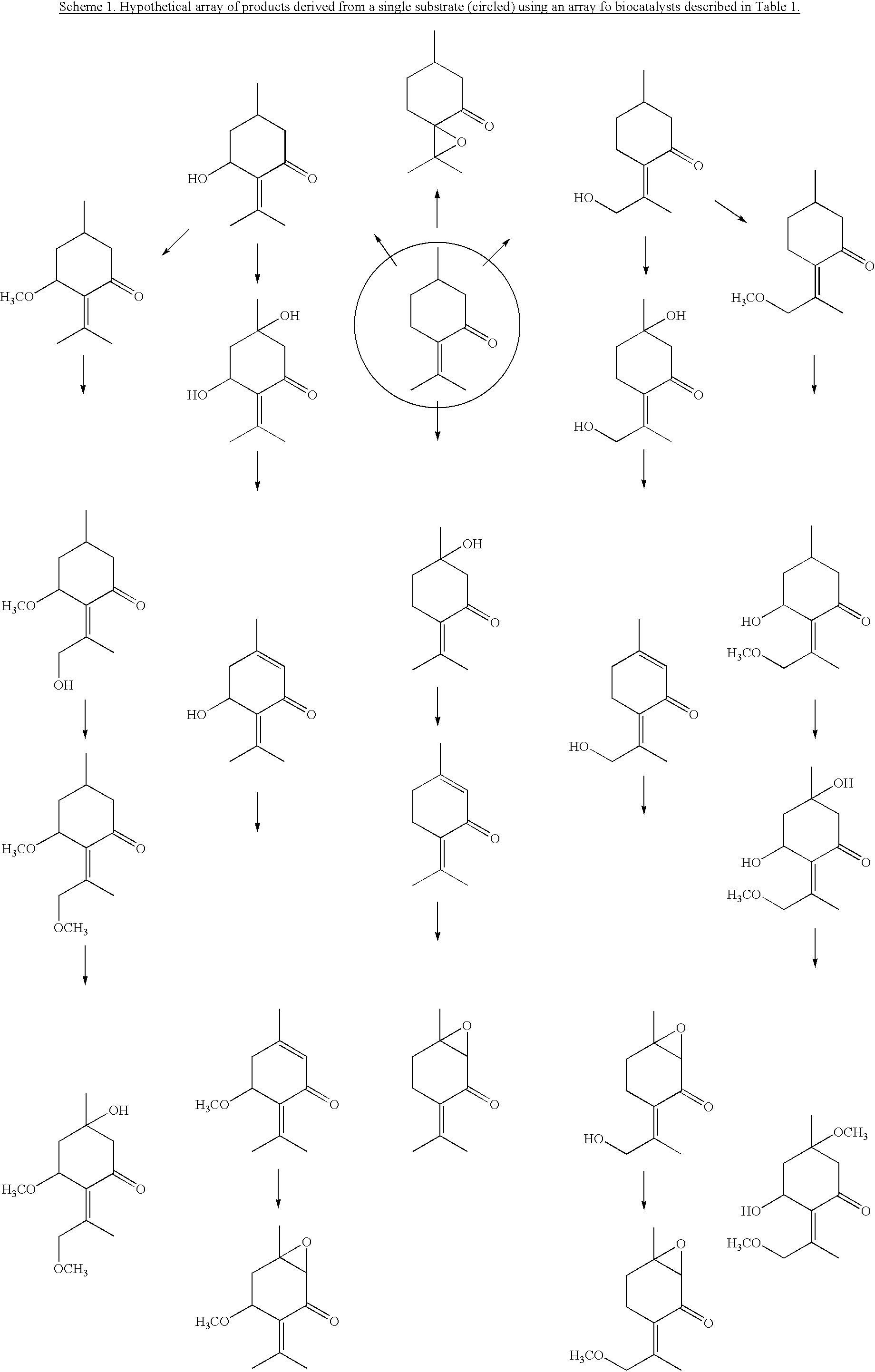

Various modifications and alterations of this invention will become apparent to those skilled in the art without departing from the scope and intent of this invention, and it should be understood that this invention is not to be unduly limited to the illustrative embodiments, conditions, or details set forth herein. The examples show specific performance of individual ingredients, but is not intended to limit the practice of the invention to only those ingredients, practices and conditions. One of ordinary skill in the relevant art comprehends these examples to be representative, not limiting.

1. A method for producing one or more biocatalysts or a combinatorial array of biocatalysts comprising the step of:
a) providing a host cell;
b) recombining at least two biotransformation genes for modifying a chemical substrate into the host cell;
c) thereby producing at least one recombinant strain comprising a biocatalysts.

2. The method according to claim 1 wherein said at least two biotransformation genes introduce two different chemical functional groups, at least one of the at least two chemical functional groups selected from the group selected from carbon to carbon bonds, hydroxylation, halogenation, cycloaddition, and amination.

3. The method according to claim 1 wherein said biotransformation genes modify functional groups according to reactions selected from the group selected of
i. reduction;
ii. oxidation;
iii. hydrolysis;
iv. replacement;
v. ring cyclization;
vi. isomerization;
vii. epimerization; and
viii. dealkylation.

4. The method according to claim 3 wherein the reactions are selected from the group consisting of:
i. reduction of carboxylic acids, aldehydes, and ketones;
ii. oxidation of alcohols, sulfites, amino groups, and thiols;
iii. hydrolysis of nitriles;
iv. replacement of amino groups with hydroxyl groups;

5. The method according to claim 1 wherein said biotransformation genes provide functional group addition of groups capable of providing catalysis for processes selected from the group consisting of acylation, glycosylation, amidation, phosphorylation, and alkyl transfer.

6. The method according to claim 1 wherein said biotransformation genes are derived from whole cells endowed with biotransformation ability as a result of genetic recombination and in vivo expression from one or both of constitutive promoter(s) and inducible promoter(s) to create whole-cell biocatalysts.

7. The method according to claim 2 wherein said biotransformation genes are derived from whole cells endowed with biotransformation ability as a result of genetic recombination and in vivo expression from one or both of constitutive promoter(s) and inducible promoter(s) to create whole-cell biocatalysts.

8. The method according to claim 3 wherein said biotransformation genes are derived from whole cells endowed with biotransformation ability as a result of genetic recombination and in vivo expression from one or both of constitutive promoter(s) and inducible promoter(s) to create whole-cell biocatalysts.

9. The method according to claim 4 wherein said biotransformation genes are derived from whole cells endowed with biotransformation ability as a result of genetic recombination and in vivo expression from one or both of constitutive promoter(s) and inducible promoter(s) to create whole-cell biocatalysts.

10. A method for the screening of cells having at least two distinct biotransforming catalysts combined therein, the method selected from the group consisting of:
a. providing multiple wells, each of the multiple wells containing cells having at least two distinct biotransforming catalysts combined therein; and
b. providing multiple wells, each of the wells containing different cells having at least two distinct biotransforming catalysts combined therein;
then allowing cells within the well to catalyze a reaction with a compound within each well, and analyzing the results of any reactions within each well to determine any functional activity of cells within each well.

11. The method of claim 10 wherein each of the multiple wells contain similar cells having at least two distinct biotransforming catalysts combined therein and different compounds are added to at least some of the multiple wells, and analyzing the results of any reactions within each well to determine any functional activity of cells within each well.

12. The method of claim 10 wherein each of the multiple wells contain different cells having at least two distinct biotransforming catalysts combined therein and similar compounds are added to at least some of the multiple wells, and analyzing the results of any reactions within each well to determine any functional activity of cells within each well.

13. The method of claim 10 wherein at least 12 wells are used as the multiple wells.

14. The method of claim 10 wherein at least 20 wells are used as the multiple wells.

15. The method of claim 10 wherein at least 40 wells are used as the multiple wells.

16. The method of claim 10 wherein at least 90 wells are used as the multiple wells.

17. A method for producing a combinatorial array of biocatalysts comprising the steps of:
a) providing a host cell;
b) recombining one biotransformation gene for modifying a chemical substrate into the host cell;
c) thereby producing at least one recombinant strain comprising a biocatalyst;
d) then inserting the at least one recombinant strain comprising a biocatalyst into at least two sections of an array of biocatalysts.

18. The method according to claim 17 wherein said at least one biotransformation gene introduces a chemical functional group selected from the group selected from carbon to carbon bonds, hydroxylation, halogenation, cycloaddition, and amination.
